(19) United States
(12) Patent Application Publication
(10) Pub. No.: US 2007/0219515 A1
(43) Pub. Date: Sep. 20, 2007

(54) Absorbent articles with lotions

(75) Inventors: Randall Glenn Marsh, West Chester, OH (US); Ekaterina Anatolyevna Ponomarenko, Bad Soden (DE); Victor Nicholas Vega, Cincinnati, OH (US); Philip Andrew Sawin, Wyoming, OH (US)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(73) Assignee: The Procter & Gamble Company, Cincinnati, OH (US)

(21) Appl. No.: 11/717,928

(22) Filed: Mar. 14, 2007

Related U.S. Application Data
(60) Provisional application No. 60/782,704, filed on Mar. 14, 2006.

(51) Int. Cl. A61F 13/15

(52) U.S. Cl. 604/359; 516/53; 604/364

(57) ABSTRACT

This application relates to absorbent articles, to be worn by a user, comprising a backsheet and in addition a topsheet, and/or barrier cuffs, and/or leg cuffs, comprising on one or more surfaces thereof that are in contact with the skin of the user, a coating composition or partial coating with a particulate material dispersed in a carrier component. More particularly, said coating composition is a lotion composition that is transferable to the wearer's skin by normal contact and wearer motion and/or body heat. Preferred articles herein have at least such a topsheet and preferred are infant (baby) diapers, including training pants, and adult incontinence devices, sanitary napkins, panty-liners and the like.

### CROSS REFERENCE TO RELATED APPLICATION

This application claims the benefit of U.S. Provisional Application No. 60/782,704, filed Mar. 14, 2006, the substance of which is incorporated herein by reference.

### FIELD OF THE INVENTION

This application relates to absorbent articles, to be worn by a user, comprising a backsheet and in addition a topsheet, and/or barrier cuffs, and/or leg cuffs, comprising on one or more surfaces thereof that are in contact with the skin of the user, a coating or partial coating of a coating composition with a particulate material dispersed in a carrier component. More particularly, said coating composition is a lotion composition that is transferable to the wearer's skin by normal contact and wearer motion and/or body heat. Preferred articles herein have at least such a topsheet and preferred are infant (baby) diapers, including training pants, and adult incontinence articles, sanitary napkins, panty-liners and the like.

The particulate material of the coating compositions herein is believed to reduce the contact of the skin with feces or menses and the adherence of the menses or feces to the skin, thereby improving the ease of BM clean up.

### BACKGROUND OF THE INVENTION

Many types of disposable absorbent products, such as diapers and sanitary napkins, are available that have a topsheet comprising a lotion, to deliver skin benefits to the skin of the user and to sometimes improve the removal of feces or menses from the skin.

In recent years the focus has been to deliver lotions to sanitary napkins and diapers that provide extra skin benefits, for example by addition of botanical ingredients or pharmaceutical ingredients to the lotions.

However, the inventors found that there is an unmet need to provide diapers and sanitary napkins with some means to reduce the adherence of menses or feces to the skin, and that further provides improved means to remove any menses or feces that does adhere to the skin.

The inventors have found that by applying particulate material to the surfaces of the absorbent article that are in contact with the skin of the user, e.g. the topsheet, reduced feces or menses adherence to the skin can be achieved and improved removal of feces or menses can be obtained. It is believe that the particles may not only form a barrier between the feces or menses and the skin, reducing thus the surface of the menses and feces that is in contact with the skin and can adhere thereto, but that, in particular when incorporated in a skin-transferable (lotion) composition, it may facilitate the removal of menses and feces that may be present on the skin, by for example acting as a lubricant.

Disposable absorbent articles, such as diapers, training pants, and catamenial devices having lotioned topsheets are known. Lotions of various types are known to provide various skin benefits, such as prevention or treatment of diaper rash. These lotions can be applied to the topsheet of absorbent articles, for example, and can be transferred to the skin of the wearer during use.

Unlike many types of disposable absorbent articles, catamenial devices such as pads and pantiliners are specifically designed to acquire menstrual fluid. Menstrual fluid differs from other exudates, such as urine, in many important properties, such as viscosity. Therefore, catamenial devices should differ in their structural components from such devices as baby diapers to be optimized for the maximum absorption of menstrual fluid.

The addition of lotion to the topsheet of absorbent articles is known to provide benefits such as easier BM clean up on babies. Likewise, lotion on topsheets is known to provide for better skin health of babies, such as the reduction of diaper rash. For example, U.S. Pat. No. 3,489,148 to Duncan et al. teaches a baby diaper comprising a hydrophobic and oleophobic topsheet wherein a portion of the topsheet is coated with a discontinuous film of oleaginous material. A major disadvantage of the diapers disclosed in the Duncan et al. reference is that the hydrophobic and oleophobic topsheets are slow in promoting transfer of urine to the underlying absorbent cores. Since the viscosity of menses is considerably greater than urine, the problems associated with Duncan et al are more profound.

One successful attempt at overcoming the problems of Duncan is disclosed in Roe et al., U.S. Pat. No. 5,968,025. Roe et al. discloses an absorbent article in which a lotion is applied to a hydrophilic topsheet (or a topsheet rendered to be hydrophilic). The hydrophilic topsheet aids in ensuring urine gushes are adequately absorbed into the underlying core, rather than running off into the sides of a baby diaper, for example.

The known attempts at applying lotions to topsheets of absorbent products have been primarily directed to baby diapers, with the benefit provided being better skin health for the bottom of the baby. Little attention has been directed to the unique problems associated with the skin of an adult woman when wearing a catamenial pad. The skin of the vulvar area of an adult woman is very different than that of a baby's bottom (or buttock skin in general), and the lotion needs are very different. For example, rather than being concerned with diaper rash, a menstruating woman is more concerned about hygiene, that is, reducing the amount of menses remaining on the skin and hair after use of a sanitary pad.

The aforementioned attempts at providing a lotion on a topsheet of an absorbent article have focused on the lotion/topsheet characteristics necessary to handle a gush of urine in a relatively short amount of time. However, for catamenial devices, the fluid insult has very different characteristics, in the context of physio-chemical properties (e.g., viscosity, fluid dynamics, etc.) and in the volume and in the time to be absorbed. For example, menstrual flow typically consists of two patterns. One of these is “trickle” flow, which varies from 0.1 to 2 ml per hour. The second pattern is “gush” flow which varies from a few ml in volume delivered over a few seconds. Gush flow can result from an accumulation of menses pooling in the vagina which can then exit the body upon a change in position, such as a transition from sitting to standing. In any event, even with gush flow, the total amount of fluid required to be absorbed into the core in a given time is much less than that required by other absorbent products, such as baby diapers, for example. One practical result is that catamenial devices, rather than needing to be designed to handle gushing fluid, more typically handle fluid through a “blotting” effect.

Accordingly, there is a continuing need for a catamenial device having improved fluid handling such that more menses enter into and remain in the device, and less on the skin and hair of the wearer.

However, the inventors found that there is an unmet need to provide sanitary napkins with some means to reduce the adherence of menses to the body, and that provides improved means to remove any menses that does adhere to the body, and that further provides an improved comfort to the wearer.

The inventors have found that by applying particulate material to the surfaces of the absorbent article that are in contact with the body of the user, e.g. the topsheet, reduced feces or menses adherence to the body can be achieved and improved removal of feces or menses can be obtained. It is believed that the particles form a physical barrier between the feces or menses and the body, reducing thus the surface of the feces or menses that is in contact with the body and can adhere thereto, but that, in particular when incorporated in a skin-transferable (lotion) composition, it may facilitate the removal of feces or menses that may be present on the body, by for example acting as a lubricant. Furthermore, when formulated to deliver a non-uniform distribution of particulates on the topsheet of the absorbent article, it may further increase the efficiency of lotion transfer to the body to promote an even further cleaner body. Alternatively this approach, while being more efficient will also be more cost effective.

### SUMMARY OF THE INVENTION

The present invention relates to an absorbent article, to be worn by a user, comprising a backsheet and a topsheet and/or leg cuffs and/or barrier cuffs, said topsheet, leg cuffs and/or barrier cuffs having a first surface, and/or an overwrap or string of a tampon, comprising a lotion composition can be applied to at least a portion of at least the body facing surface of the topsheet and/or leg cuffs and/or barrier cuffs, said lotion composition comprises a solid or semi-solid, or semi-fluid carrier component and therein dispersed a particulate material.

The particulate material is typically inert and insoluble in water and preferably insoluble in ethanol, but dispersible in water and/or ethanol.

The composition is typically a lotion composition as described herein.

The particulate material typically has prior to application to the topsheet or cuffs (and typically both prior to incorporation into the composition and once incorporated into the composition but prior to application to the article) a mean particle size from 1 nm to 2 mm, preferably a mean particle size from 20 microns to 1000 microns.

The composition on the article, i.e. once in the form of the coating, is typically solid at 20° C.

### DETAILED DESCRIPTION OF THE DRAWINGS

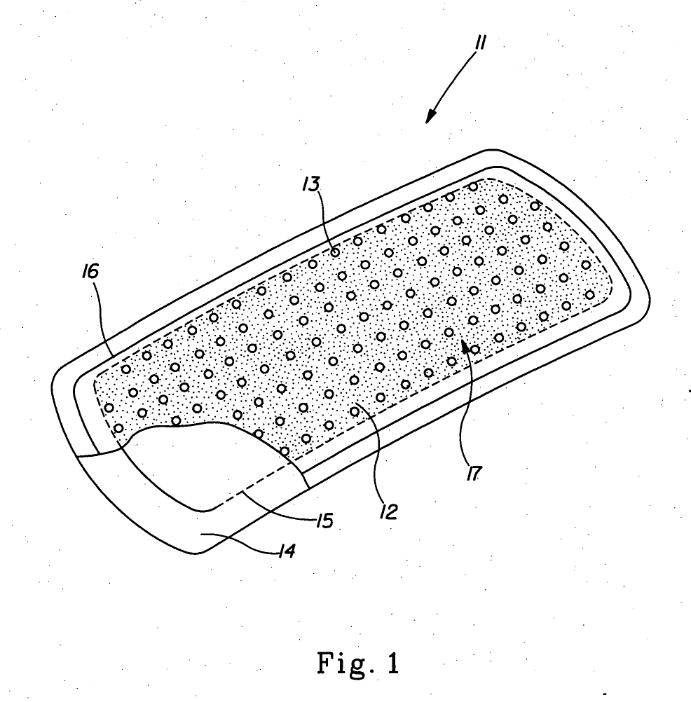

FIG. 1 shows an apertured panty liner 11 comprising a topsheet 12 containing apertures 13, a liquid impervious backsheet 14 bonded to the topsheet 12 along a sealing line 16, and an absorbent core 15. The topsheet 12 also contains a skin care composition 17 disposed on its top surface, wherein the skin care composition 17 is distributed onto the topsheet 12 such that it remains on the top surface and not within the apertures 13 for effective transfer of the skin care composition 17 onto the skin of a wearer.

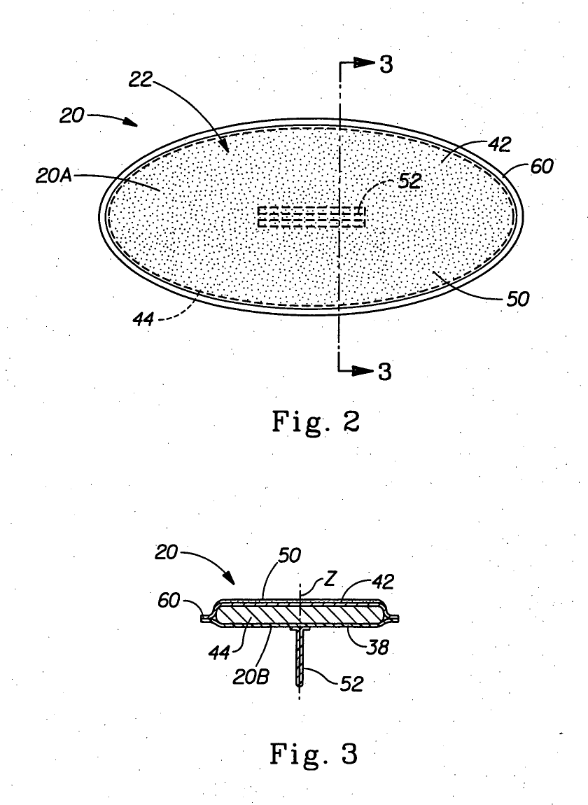

FIG. 2 shows an interlabial product containing a skin care composition of the present invention. FIG. 3 is a cross sectional view of the interlabial product shown in FIG. 3, taken along line 4-4 of FIG. 2. The interlabial product 20 as shown in FIGS. 2-3 has a body-contacting side 20A and a garment surface 20B. The interlabial product comprises a pad-like main body portion 22 and an optional placement and removal tab 52 which is joined to the underside 20B of the main body portion 22 to provide the overall interlabial product with a “T”-shaped cross-sectional configuration. As shown in FIGS. 2-3 the main body portion 22 comprises a topsheet 42, a liquid impervious backsheet 38 joined to the topsheet 42 along a seam 60, and an absorbent core 44. The skin care composition 50 is disposed on the topsheet 42. The interlabial product 20 is also suitable for use as a hemorrhoid patch.

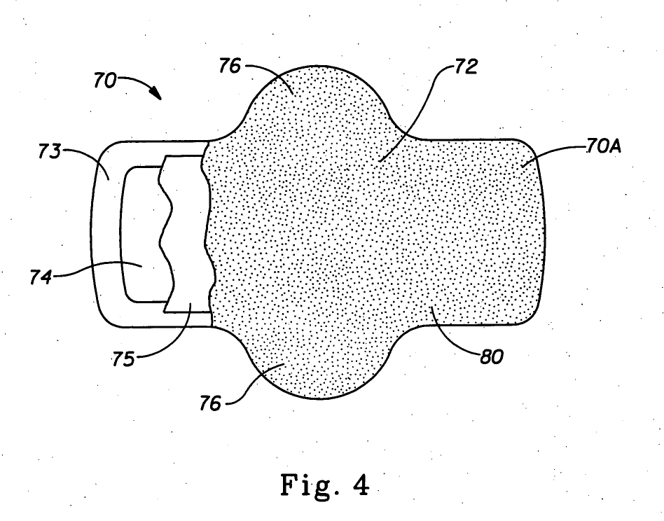

FIG. 4 shows a sanitary napkin 70 having a body-contacting surface 70A comprising a topsheet 72, a liquid impervious backsheet 73 joined to the topsheet 72, an absorbent core 74, and a fluid acquisition layer 75 to promote fluid transport to the absorbent core 74. The sanitary napkin 70 may also be provided with additional features commonly found in napkins, including “wings” or “flaps” such as wings 76. The topsheet 72 portion of the sanitary napkin 70 has a skin care composition 80 disposed onto the topsheet.

### DETAILED DESCRIPTION OF THE INVENTION

Herein, “comprise” and “include” mean that other elements and/or other steps which do not affect the end result can be added. Each of these terms encompasses the terms “consisting of” and “consisting essentially of”.

Herein, “component fibers” refers to fibers which form a material (i.e., a topsheet material, a backsheet material, or an absorbent core material) of the absorbent article. The component fibers can be natural fibers (e.g., wood or cotton fibers), modified natural fibers, synthetic fibers (e.g., thermoplastic fibers such as polyester, polypropylene, or polyethylene fibers), or combinations thereof.

Preferred component fibers for the topsheet are thermoplastic fibers. Preferred component fibers for the absorbent core are natural fibers (e.g., wood or cotton fibers) or a mixture of natural fibers and thermoplastic fibers.

Herein, “body surface” refers to surfaces of absorbent articles and/or their component materials which face the body of the wearer, while “garment surface” refers to the opposite surfaces of the absorbent articles and/or their component materials that face away from the wearer when the absorbent articles are worn. Absorbent articles and components thereof, including the topsheet, backsheet, absorbent core, and any individual layers of their component materials, have a body surface and a garment surface.

Herein, “body” refers to outer layers formed by mammalian epidermal tissues including the skin and hair. The characteristics of the body tend to differ dramatically depending on the position, age, sex, and individual's nature. For example, the skin of babies and young children differs from the skin of adults, and the skin having hair differs from the non-haired skin.

As used herein “absorbent article” refers to devices which are placed against the skin of a wearer to absorb and contain the various exudates discharged from the body. As used herein “disposable” is used to describe absorbent articles which for a single use and are not intended to be laundered, restored or otherwise reused as an absorbent article after a single use. Examples of disposable absorbent articles include feminine hygiene articles such as tampons, interlabial devices, sanitary napkins and panti-liners, diapers, incontinence briefs, diaper holders, training pants, and the like. As used herein “tampon” refers to any type of absorbent structure which is inserted into the vaginal canal or other body cavities for the absorption of fluid therefrom. The basic tampon structures are described in U.S. Pat. Nos. 1,926,900 issued to Haas on Sep. 12, 1933; U.S. Pat. No. 1,946,911 issued to Haas on Jul. 3, 1934; and U.S. Pat. No. 3,322,123 issued to Giswold, et al. on May 30, 1967. As used herein “interlabial absorbent article” refers to an absorbent device that is insertable into the interlabial space of a female wearer for catamenial purposes, incontinence barrier, or both. Suitable interlabial absorbent articles are disclosed in, e.g., U.S. Pat. No. 5,762,644 entitled “Toilet-Disposable Absorbent Interlabial Device” issued to Osborn, et al. on Jun. 9, 1998; PCT Publication No. WO 98/29078 entitled “Thin Comfortable Interlabial Absorbent Structure” published in the name of Osborn, et al. on Jul. 9, 1998; U.S. Pat. Des. 404,814 entitled “Interlabial Absorbent Device” issued to Mayer on Jan. 26, 1999; U.S. Pat. No. 6,270,486 entitled “Absorbent Interlabial Device” issued to Brown, et al. on Aug. 7, 2001. As used herein, the terms “panty liner” or “panti-liner” refer to absorbent articles that are less bulky than sanitary napkins which are generally worn by women between their menstrual periods. Suitable absorbent articles are disclosed in, e.g., U.S. Pat. No. 4,738,676 entitled “Pantiliner” issued to Osborn on Apr. 19, 1988. As used herein “diaper” refers to an absorbent generally worn by infants, and incontinent persons that is worn about the lower torso of the wearer. Suitable diapers are disclosed in, e.g., U.S. Pat. No. 3,860,003 issued to Buell on Jan. 14, 1975; U.S. Pat. No. 5,151,092 issued to Buell et al. on Sep. 29, 1992; U.S. Pat. No. 5,221,274 issued to Buell et al. on Jun. 22, 1993; and U.S. Pat. No. 5,554,145 issued to Roe et al. on Sep. 10, 1996. As used herein “incontinence article” refers to pads, undergarments, inserts for absorbent articles, capacity boosters for absorbent articles, briefs, bed pads, and the like regardless of whether they are worn by adults or other incontinent persons. Suitable incontinence articles are disclosed in, e.g., U.S. Pat. No. 4,253,461 issued to Strickland, et al. on Mar. 3, 1981; U.S. Pat. Nos. 4,597,760 and 4,597,761 issued to Buell; the above-mentioned U.S. Pat. No. 4,704,115; U.S. Pat. No. 4,909,802 issued to Ahr, et al.; U.S. Pat. No. 4,964,860 issued to Gipson, et al. on Oct. 23, 1990; and PCT Publication No. WO 92/11830 published by Noel, et al. on Jul. 23, 1992. As used herein “training pants” refers to disposable garments having fixed sides and leg openings. Training pants are placed in position on the wearer by inserting the wearer's legs into the leg openings and sliding the training pant into position about the wearer's lower torso. Suitable training pants are disclosed in, e.g., U.S. Pat. No. 5,246,433, issued to Hasse, et al. on Sep. 21, 1993.

The absorbent article has two surfaces, a liquid pervious body surface (or “body-contacting surface”) and a liquid impervious garment surface (or “garment-contacting surface”). The body surface of the absorbent article is intended to be worn adjacent to the body of the wearer. The garment surface of the absorbent article is on the opposite side and is intended to be placed adjacent to the wearer's undergarments when the absorbent article is worn.

The term “joined”, as used herein, encompasses configurations in which an element is directly secured to another element by affixing the element directly to the other element; configurations in which the element is indirectly secured to the other element by affixing the element to intermediate member(s) which in turn are affixed to the other element; and configurations in which one element is integral with another element, i.e., one element is essentially part of the other element.

The term “longitudinal”, as used herein, refers to a line, axis or direction in the plane of the sanitary napkin that is generally aligned with (e.g., approximately parallel to) a vertical plane which bisects a standing wearer into left and right body halves when the sanitary napkin is worn. The terms “transverse” or “lateral” used herein, are interchangeable, and refer to a line, axis or direction which lies within the plane of the sanitary napkin that is generally perpendicular to the longitudinal direction.

As used herein, the terms “migrate”, “migration”, or “migrating” mean the skin care composition moves from one place to another place by way of movement on a material or permeation through an intervening material.

As used herein, the term “transfer” when used in the context of the skin care composition, refers to the skin care composition moving from one area of the absorbent article to another area on the absorbent article not by way of migration but by way of direct contact with the skin care composition, such as in a blotting effect.

“Coating” on the topsheet, barrier cuff or leg cuff as used herein means that said topsheet, barrier cuff or leg cuff comprises at least a partial layer of the composition on at least part of its outer surface so that at least part of the composition may contact the skin of the user in use.

As used herein, the term “particulate material” refers to a component of the composition that is insoluble or non-molecularly dispersible or non-reactive biologically in the composition prior to applying this composition to the absorbent article and that remains in particulate form when applied to the absorbent article. It includes all type of particulate forms such as granules, beads, spheres, micro-spheres, powders, as known in the art.

“Carrier component” when used herein means any medium that is capable of dispersing the particulate material.

As used herein, the terms “emulsifier” or “solubilizer” refer to a component that reduces the tendency of one or more of the other components in a lotion composition to separate into an individual bulk phase.

The absorbent articles herein comprise a backsheet. This backsheet may typically be a liquid impervious backsheet, as known in the art. In one embodiment, the liquid impervious backsheet comprises a thin plastic film such as a thermoplastic film having a thickness of about 0.01 mm to about 0.05 mm. Suitable backsheet materials comprise typically breathable material, which permit vapors to escape from the absorbent article while still preventing exudates from passing through the backsheet. Suitable backsheet films include those manufactured by Tredegar Industries Inc. of Terre Haute, Ind. and sold under the trade names X15306, X10962 and X10964. The backsheet, or any portion thereof, may be elastically extendable in one or more directions.

The absorbent articles herein typically have a topsheet and the diapers or adult incontinence products herein typically have also leg cuffs and/or barrier cuffs. The sanitary napkins or panty-liners herein may comprise as cuffs (with the coating/composition as described herein) so-called wings, for example attachment to underwear of the user.

The topsheet herein may also be a topsheet that has one or more openings that are large enough to let feces or menses pass to a void space underneath said secondary topsheet, also referred to as anal or vaginal cuff.

The backsheet may be directly or indirectly attached (or joined) to the topsheet herein and/or the barrier and/or leg cuffs herein. The absorbent article typically also comprises an absorbent core, which may be directly or indirectly attached to the backsheet and/or topsheet.

It may be highly preferred that the longitudinal side edges of the topsheet and backsheet are directly attached to one another, but that the longitudinal edges of the topsheet and the core are not attached to one another.

The above-referred to attachment may be done by any means known in the art, e.g. by use of adhesive heat bonds, pressure bonds, ultrasonic bonds, dynamic mechanical bonds, or any other suitable attachment means or combinations of these attachment means as are known in the art.

A suitable topsheet may be manufactured from a wide range of materials, including woven or non-woven webs of natural fibers (e.g., wood or cotton fibers) or synthetic fibers (e.g., polyester, polyethylene and/or polypropylene fibers), or a combination of natural and synthetic fibers. If the topsheet includes fibers, the fibers may be, for example, spun bond, carded, wet-laid, melt blown, hydro entangled, or otherwise processed as is known in the art. Preferred are meltblown and/or spunbond fibrous webs or laminates thereof. The topsheet may be hydrophilic e.g. to improve passage of urine. It may be apertured and then, it may also be hydrophobic, e.g. it may be an apertured formed film or plastic film with apertures.

Suitable topsheet materials may be compliant, soft feeling, and non-irritating to the wearer's skin.

The absorbent article herein may have one or more pairs of (elasticated) leg cuffs, including so-called side panels or wings, and/or (elasticated) barrier cuffs that provide improved containment of liquids and other body exudates, and these cuffs may in one embodiment comprise the coating composition described herein. Suitable cuffs are described in for example U.S. Pat. Nos. 3,860,003; 4,808,178 and 4,909; 4,695,278 and 4,795,454.

The cuffs may also be made of nonwoven materials as described above and they are preferably hydrophobic.

Suitable materials for the topsheet and/or cuffs include webs comprising spunbond layers (S) and meltblown layer(s) (M), whereby the surfaces of the web are formed by spunbond layer(s).

In one embodiment, the webs may have a relatively high basis weight, for example more than 25 gram/m² (gsm). Suitable webs include, for example, 34 gsm SMMS (whereby 12 gsm meltblown and 5 gsm spunbond); 34 gsm SMMS (whereby 10 gsm meltblown and 7 gsm spunbond); 30 gsm SMMS (whereby 10 gsm meltblown and 5 gsm spunbond); 30 gsm SMMS (whereby 8 gsm meltblown and 7 gsm spunbond); 34 gsm SMS (whereby 20 gsm meltblown and 7 gsm spunbond), or, for example, webs comprising two layers of 17 gsm SMMS.

The absorbent core may comprise any absorbent material which is generally compressible, conformable, non-irritating to the wearer's skin, and capable of absorbing and retaining urine or other bodily exudates, such as comminuted wood pulp, creped cellulose wadding; melt blown polymers, including coform; chemically stiffened, modified or cross-linked cellulosic fibers; tissue, including tissue wraps and tissue laminates; absorbent foams; absorbent sponges; super absorbent polymers; absorbent gelling materials; or any other known absorbent material or combinations of materials; preferred may be absorbent cores which have an absorbent storage layer which comprises more than 80% by weight of the absorbent core content (e.g., excluding core wrap) of absorbent gelling material, and which is preferably free of airfelt.

The diapers may comprise a front and back waist band and/or a fastening system, typically joined to the waistband, as known in the art. Preferred fastening systems comprise fastening tabs and landing zones, wherein the fastening tabs are attached or joined to the back region of the diaper and the landing zones are part of the front region of the diaper.

The sanitary napkins and/or panty-liners herein may preferably comprise a fastening means comprised by the backsheet and/or by the wings (cuffs), as described above. Preferred are adhesive attachment means that are present on or attached to at least the backsheet.

### Compositions and Coating Thereof

The composition that is applied to the absorbent articles herein comprises a particulate material and a carrier component that is at least capable of dispersing the particulate material herein, e.g. in order to apply said particulate material evenly to the absorbent article (i.e. the outer surfaces of the topsheet, leg cuffs and/or barrier cuffs of the absorbent article herein). It is thereto a composition that comprises a carrier component that is (at least) flowable (e.g. liquid or semi-liquid) at suitable process conditions, e.g. above 50° C. or above 60° C. or above 80° C. (or optionally above 100° C.). The carrier component and particulate material should be selected such that the particulate material does not dissolve in the carrier component and typically such that the carrier component does not change the particle size or particle size distribution of the particles and/or otherwise alters the particle's properties.

The coating formed may be a layer or partial layer on the surface of the topsheet or cuffs but it may also impregnate the topsheet or cuffs, provided it is preferably at least partially present on the outer surface of the topsheet or cuffs. It may cover or coat a part of said surface or the whole surface. It may for example be applied only in a specific pattern, such that the surface has region(s) where the coating is present and region(s) where no coating is present. For example, it may be applied in one or more longitudinal stripes, and/or only in the centre zone of the topsheet or cuffs, e.g. the central 30%-70% of the surface area of the topsheet or cuffs.

The composition herein is preferably a lotion composition that provides skin care benefits, and optionally also cleaning benefits to the skin.

The (lotion) compositions of the present invention are typically solid, or more often semisolid, at 20° C., i.e. at ambient temperatures. By “semisolid” is meant that the lotion composition has a rheology typical of pseudoplastic or plastic fluids. When no shear is applied, the compositions can have the appearance of a semi-solid but can be made to flow as the shear rate is increased. This may be due to the fact that, while the lotion composition contains primarily solid components, it also includes some minor liquid components.

The (lotion) compositions of the present invention are at least semi-solid at room temperature to minimize lotion migration. In addition, the lotion composition's carrier component preferably has a final melting point (100% liquid) above potential “stressful” storage conditions that can be greater than 45° C.

In one embodiment, the composition is such that 3% to 25% by weight is liquid at room temp. (20° C.) and 25% to 70% or even 30% to 90% is liquid at body temperature (37° C.)

When applied to (the outer surface of) the topsheets or cuffs, the lotion compositions of the present invention are transferable to the wearer's skin by normal contact, wearer motion, and/or body heat.

### Particulate Material

An essential component of the coating composition and the coating of the present invention is a particulate material. The particulate material is particulate during application onto the absorbent article and once applied to the absorbent article. It typically has the same chemical and physical properties prior to incorporation into the composition and/or during incorporation into the composition as once applied to the absorbent article.

The particulate material is also such that it remains particulate when in contact with the skin and/or when in contact with urine, menses or feces.

Hence, the particulate material is water-insoluble and it has a melting temperature of more than the processing temperature of the composition, as described above.

The particulate material may have in the composition to be applied or in the applied coating any mean particle size between 1 nanometer to 2 mm, preferably between 1 nanometer to 500 micrometers, more preferably between 0.1 micrometer to 2 mm, and still more preferably between 50 nanometers to 1 micrometer, or any range or individual value within any of the ranges set forth herein. Preferably, the minimum mean particle size is at least 0.1 micrometer or preferably at least 1 micrometer, or preferably at least 10 micrometers, or more preferably at least 20 micrometers, and preferably up to about 500 micrometers or in some embodiments up to about 100 micrometers, and further in other embodiments up to about 30 micrometers. In one embodiment, it may be preferred that the composition to be applied and/or the applied coating comprises particles whereof less than 25% of the particles have an equivalent diameter of greater than 100 microns. In another embodiment, it may be preferred that the composition to be applied and/or the applied coating comprises particles whereof less than 25% of the particles have an equivalent diameter of less than 5 microns. In yet another embodiment, it may be preferred that the composition to be applied and/or the applied coating comprises particles whereof less than 25% of the particles have an equivalent diameter of less than 100 microns.

The particle material may be present in the composition at a level from 0.05% to 25% (by weight of the composition), preferably from 0.05% to 15%, more preferably from 0.05% to 5%, or from 0.1% to 25%, or more preferably from 0.25% to 20%, but typically from 0.5% to 10% or even up to 5% by weight.

Suitably, the particles may have a density between about 0.5 gram/cm³ and about 2.5 gram/cm³. Preferably, the density is between about 0.5 gram/cm³ and about 2.0 gram/cm³, and more preferably between 0.8 gram/cm³ and about 1.5 gram/cm³. In one embodiment, the density may preferably be less than about 1 gram/cm³ so as to minimize particle settling and the density is greater than about 0.8 gram/cm³ so as to minimize particle floatation.

In one embodiment, the composition may comprise inorganic particles, including alumina silicates, silicates, silicas, mica and/or talc. Clays may also be used. However, in the present invention it may be preferred that the particulate material is an organic material.

Preferably, the particles are a non-active and/or non-reactive material.

The particles may be porous, but it may be preferred that the particles are non-porous.

The particles may have any shape, but preferably they have a smooth surface, and they may be preferably spherical or plate-like particles.

The particles may comprise a coating agent on its surface or part thereof, for example a surfactant, for example to change its properties, e.g. hydrophilicity. The particles, in particular when they are oleofinic, may include a melt-additive, which is added during the manufacturing of the particles.

Suitable materials include but are not limited to: polystyrene particles, polypropylene and/or polyethylene (co)polymer particles, polytetraflouorethylene particles, polymethylsilsequioxane particles, nylon particles.

Suitable commercially available particulate materials include but are not limited to: polyethylene particles, available from Honeywell International of Morristown, N.J. under the trade name ACUMIST; polymethyl methacrylate particles (microspheres), available from KOBO of South Plainfield, N.J. as BPA; lactone cross polymer particles (microspheres), available from KOBO as BPD; nylon 12 particles (microspheres), available from KOBO as NYLON SP; polymethylsilsesquioxane particles (microspheres), available from KOBO as TOSPEARL; cellulose particles (microspheres), available from KOBO as CELLO-BEADS; polytetrafluoroethylene powders, available from Micro Powders, Inc. of Tarrytown, N.Y. as MICROSLIP; blends of natural wax and micronized polymers as are available form Micro Powders as MICROCARE and particles of a copolymer of vinylidene chloride, acrylonitrile and methylmethacrylate available as EXPANCEL from Expancel, Inc. of Duluth, Ga.

Micronized waxes, such as are available from Micro Powders as MICROEASE may also be incorporated.

Preferred are polyolefin particles (powders) as are available from Equistar Chemical Corp. Houston, Tex. as MICROTHENE. Particularly preferred is MICROTHENE FN510-00 from Equistar.

### Lotion Compositions

As described above, the compositions of the present invention are preferably lotion composition and preferably skin care lotion compositions.

The lotion compositions of the present invention may comprise a body treatment agent, a carrier, and an immobilizing agent(s) for the carrier, and optionally a hydrophilic surfactant(s) and/or rheology modifier and/or other optional components.

The lotion compositions of the present invention can comprise a select combination of body treatment agents such as hexamidine, zinc oxide, and niacinamide which are highly effective in the prevention and treatment of erythema, malodor, and bacterial skin disorders, especially when these lotion compositions are administered to the body from application on absorbent articles.

The term “body treatment agent” as used herein to include treatments effective on skin or hair of mammalian bodies, and refers to materials that when transferred to the body are capable of preventing, reducing, and/or eliminating occurrences of skin disorders, particularly skin disorders associated with erythema, malodor, and bacterial infections. The term “skin disorders” as used herein refers to symptoms associated with irritating, acute, or chronic skin abnormalities. Examples of such symptoms include, but are not limited to, itching, inflammation, rash, burning, stinging, redness, swelling, sensitivity, sensation of heat, flaking/scaling, malodor, and the like. The term “ambient conditions” as used herein refers to surrounding conditions at about one atmosphere of pressure, at about 50% relative humidity, and at about 25° C.

The lotion compositions of the present invention can comprise, consist of, or consist essentially of the elements and limitations of the invention described herein, as well as any of the additional or optional ingredients, components, or limitations described herein. All percentages, parts and ratios are by weight of the total composition, unless otherwise specified. All such weights as they pertain to listed ingredients are based on the specific ingredient level and, therefore, do not include carriers or by-products that may be included in commercially available materials, unless otherwise specified.

I. Skin Treatment Agents

The lotion compositions of the present invention comprise relatively low concentrations of a select combination of skin treatment agents that are capable of reducing and eliminating the occurrence of skin disorders that can result from contact between the skin and moisture-laden air, skin disorders resulting from prolonged moist human tissue that can occur from the skin being exposed to moisture or other body exudates, and/or skin disorders that are generated from contact between the skin and microbial or bacterial agents. The phrase “select combination of body treatment agents” refers to the following combinations: a. hexamidine, zinc oxide, and niacinamide; b. hexamadine and zinc oxide; and c. hexamadine and niacinamide.

Surprisingly, the select combination of body treatment agents can be included at low individual concentrations, relative to their use in the prior art, and still be effective. For example, the lotion compositions of the present invention can include hexamidine at a concentration of about 0.1% or less by weight, zinc oxide at a concentration of about 1% or less by weight, and niacinamide at a concentration of about 2% or less by weight to achieve equal or superior benefits in the prevention and/or treatment of skin disorders as compared to known lotion compositions that generally comprise these skin treatment agents at higher levels. Similarly, the total effective concentration of the select combination of body treatment agents in the compositions of the present invention are also relatively low. The total concentration of the select combination of body treatment agents ranges from about 0.002% to about 10%, preferably from about 0.01% to about 5%, more preferably from about 0.1% to about 2% by weight of the lotion composition.

A. Hexamidine: The lotion compositions of the present invention comprise hexamidine body treatment agent at concentrations ranging from about 0.001% to about 0.1%, from about 0.005% to about 0.1%, or even from about 0.01% to about 0.1% by weight of the composition. The hexamidine body treatment agent suitable for use herein include those aromatic diamines which generally conform to the following formula:

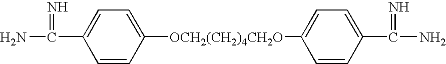

These aromatic diamines are referred to as 4,4′-[1,6-Hexanediylbis(oxy)]bisbenzenecarboximidamide; 4,4′-(hexamethylenedioxy) dibenzamidine; and 4,4′-diamidino-α,ω-diphenoxyhexane. The most popular employed form of hexamidine is the general category of hexmidine salts, which include acetate, salicylate, lactate, gluconate, tartarate, citrate, phosphate, borate, nitrate, sulfate, and hydrochloride salts of hexamidine. Specific nonlimiting examples of hexamidine salts include hexamidine isethionate, hexamidine diisethionate, hexamidine hydrochloride, hexamidine gluconate, and mixtures thereof. Hexamidine isethionate and hexamidine diisethionate are β-hydroxyethane sulfonate salts of hexamidine which are preferred for use herein as a skin treatment agent in the prevention and/or treatment of skin disorders. Hexamidine diisethionate is the most preferred hexamidine compound suitable for use as the skin treatment agent herein and is available from Laboratories Serolobilogiques (Pulnoy, France) and the Cognis Incorporation (Cincinnati, Ohio) under the tradename ELASTAB HP100.

Hexamidine compounds are known as effective body treatment agents that can control microbial growth that can lead to irritating and itching skin disorders and odor discomfort. Therefore, these skin treatment agents are often referred to as antimicrobial agents. As used herein the term “antimicrobial agents” refer to materials which function to destroy or suppress the growth or metabolism of microbes, and include the general classification of antibacterial, antifungal, antiprotozoal, antiparasitic, and antiviral agents.

It has been found, however, that a low concentration (about 0.1% or less by weight) of hexamidine provides for improved reduction and/or prevention of skin irritating infections, especially when a low amount of hexamidine is combined with a low concentration of other antimicrobial agents such as zinc oxide and/or niacinamide. This combination of hexamidine and zinc oxide and/or niacinamide can be administered topically and internally at a total concentration less than an effective amount of an applied dosage of these individual compounds. As used herein the term “effective amount” refers to an amount with provides a therapeutic benefit with minimal or no adverse reaction in the reduction and/or prevention of any noticeable or unacceptable skin abnormality which causes irritating, acute, or chronic symptoms including itching and inflammation.

Other aromatic diamines are also suitable for use as a skin treatment agent herein. Such compounds include butamidine and derivatives thereof including butamidine isethionate; pentamidine and derivatives thereof including pentamidine isethionate and pentamidine hydrochloride; dibromopropamidine and derivatives thereof including dibromopropamidine isethionate; stilbamidine and derivatives thereof including hydroxystilbamidine, stilbamidine dihydrochloride, and stilbamidine isethionate; diaminodiamidines and derivatives thereof; and mixtures thereof.

B. Zinc Oxide: The lotion compositions of the present invention comprise zinc oxide body treatment agent at concentrations ranging from about 0.001% to about 10%, preferably from about 0.005% to about 5%, more preferably from about 0.005% to about 2%, most preferably from about 0.01% to about 1% by weight of the composition. The zinc oxide body treatment agent can be included in the compositions as an individual zinc oxide compound or a combination of zinc oxides, provided that the individual or combined zinc oxide can readily combine with the hexamidine and niacinamide body treatment agents to provide antimicrobial benefits.

Niacinamide and nicotinic acid are also known as Vitamin B₃ and Vitamin B₅, whereas niacinamide is the commonly used active form. Niacinamide derivatives including salt derivatives are also suitable for use herein as a body treatment agent. Nonlimiting specific examples of suitable niacinamide derivatives include nicotinuric acid and nicotinyl hydroxamic acid.

The niacinamide body treatment agent can also be included in the composition as acidified niacinamide compounds. The process of acidifying niacinamide compounds is within the gambit of those skilled in the art, wherein one such technique involves dissolving niacinamide in an alcohol solution, adding while stirring an equal molar amount of a fatty acid such as stearic acid (e.g., mixing 1 part niacinamide to 2.4 parts stearic acid), and then air drying the mixture until the alcohol evaporates. A suitable stearic acid compound that can be used in the process of acidifying niacinamide is stearic acid sold under the tradename Emersol® 150 which is available from the Cognis Corporation.

Examples of the above niacinamide compounds are well known in the art and are commercially available from a number of sources, for example, the Sigma Chemical Company (St Louis, Mo.); ICN Biomedicals, Incorporation (Irvin, Calif.); Aldrich Chemical Company (Milwaukee, Wis.); and Em Industries HHN (Hawthorne, N.Y.).

D. Optional Components: Nonlimiting examples of optional suitable body treatment actives useful in the present invention include allantoin; aluminum hydroxide gel; calamine; cysteine hydrochloride; racemic methionine; sodium bicarbonate; Vitamin C and derivatives thereof; protease inhibitors including serine proteases, metalloproteases, cysteine proteases, aspartyl proteases, peptidases, and phenylsulfonyl fluorides; lipases; esterases including diesterases; ureases; amylases; elastases; nucleases; guanidinobenzoic acid and its salts and derivatives; herbal extracts including chamomile; and mixtures thereof. Guanidinobenzoic acid and its salts and derivatives are more fully described in U.S. Pat. No. 5,376,655, issued to Imaki et al. on Dec. 27, 1994. These other suitable skin treatment actives are typically included at concentrations ranging from about 0.001% to about 10% by weight of the lotion composition.

Furthermore, one or more optional components known or otherwise effective for use in lotion compositions may be included provided that the optional components are physically and chemically compatible with the essential body treatment and carrier components, or do not otherwise unduly impair product stability, aesthetics, or performance. Such optional components are typically included at concentrations ranging from about 0.001% to about 20% by weight of the compositions, and include materials such as water, skin conditioning agents, perfumes,

The zinc oxide body treatment agent suitable for use herein include those inorganic white and yellowish-white powders that conform to the formula ZnO, and that are more fully described in The Merck Index, Eleventh Edition, entry 10050, p. 1599 (1989). Some particularly useful forms of zinc oxide include those that are manufactured and commercially available in average particle size diameters that range from about 1 nm (nanometer) to about 10 μm (micrometer), alternatively from about 10 nm to about 1 μm or even from about 20 nm to about 500 nm. Surprisingly, the inventors have discovered that the use of the above mentioned, relatively small nanoparticle diameter size zinc oxide avoids undesirable skin or hair whitening that results from the transfer of the zinc oxide containing emollient from the topsheet of absorbent article to the wearer's body during product use. This is a particular benefit when the product is a panty liner, sanitary napkin, incontinence brief, or other absorbent article intended to be used by adults having hair in the region where the lotion composition will transfer.

Commercially available zinc oxides include the white zinc oxide powders sold under the tradename ULTRAFINE 350 which is commercially available from the Kobo Incorporation located in South Plainfield, N.J. Other suitable zinc oxide materials include a premix of zinc oxide and a dispersing agent such as polyhydroxystearic acid wherein this premix is available from the Uniqema Incorporation (Wilimington, Del.) under the tradename Arlecel® P100; and a premix of zinc oxide and an isononyl isononanoate dispersing agent which is available from the Ikeda Incorporation (Island Park, N.Y.) under the tradename Salacos® 99.

C. Niacinamide: The lotion compositions of the present invention comprise niacinamide body treatment agent as an individual niacinamide or as a combination of niacinamides at a total niacinamide concentration ranging from about 0.01% to about 10%, preferably from about 0.05% to about 5%, more preferably from about 0.2% to about 2% by weight of the lotion composition. The niacinamide body treatment agent provides for skin conditioning benefits as well as providing for increased efficacy of the body treatment agents in controlling skin disorders.

Nonlimiting examples of niacinamide body treatment agents suitable for use in the lotion compositions of the present invention include those niacinamide compounds that are amide derivatives of nicotinic acid, and that generally conform to the following formula:

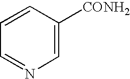

deodorants, opacifiers, astringents, preservatives, emulsifying agents, film formers, stabilizers, proteins, lecithin, urea, colloidal oatmeal, pH control agents, and other Monographed materials that are deemed safe by the U.S. Food and Drug Administration (FDA) under 21 C.F.R. §347 for use on human skin. Other optional components for use in the lotion compositions of the present invention include fats or oils, or essential oils. These oils can be present at concentrations ranging from about 0.0001% to 10% by weight of the compositions, and include materials such as Anise Oil, Apricot Kernel Oil, Avocado Oil, Balm Mint Oil, Babassu Oil, Borage Seed Oil, Butter, Bee Balm Oil, Birch Oil, Bitter Almond Oil, Bitter Orange Oil, C10-C18 Triglycerides, C12-C18 Acid Triglyceride, Camellia Oil, Calendula Oil, California Nutmeg Oil, Canola Oil, Caprylic/Capric/Lauric Triglyceride, Caprylic/Capric/Linoleic Triglyceride, Caprylic/Capric/Stearic Triglyceride, Caprylic/Capric Triglyceride, Caraway Oil, Carrot Oil, Cashew Nut Oil, Castor Oil, Chamomile Oil, Chemy Pit Oil, Chia Oil, Cinnamon Oil, Cloveleaf Oil, Clove Oil, Cocoa Butter, Coconut Oil, Cod Liver Oil, Coriander Oil, Corn Germ Oil, Corn Oil, Cottonseed Oil, Cypress Oil, Epoxidized Soybean Oil, Evening Primrose Oil, Eucalyptus Oil, Fennel Oil, Gardenia Oil, Geranium Oil, Ginger Oil, Glyceryl Triacetyl Hydroxystearate, Glyceryl Triacetyl Ricinoleate, Glycosphingolipids, Grape Seed Oil, Grapefruit Oil, Hazelnut Oil, Human Placental Lipids, Hybrid Safflower Oil, Hybrid Sunflower Seed Oil, Hydrogenated Castor Oil, Hydrogenated Coconut Oil, Hydrogenated Cottonseed Oil, Hydrogenated C2-C1 Triglycerides, Hydrogenated Fish Oil, Hydrogenated Lard, Hydrogenated Menhaden Oil, Hydrogenated Mink Oil, Hydrogenated Orange Roughy Oil, Hydrogenated Palm Kernel Oil, Hydrogenated Palm Oil, Hydrogenated Peanut Oil, Hydrogenated Shark Liver Oil, Hydrogenated Soybean Oil, Hydrogenated Tallow, Hydrogenated Vegetable Oil, Hyptis Oil, Juniper Oil, Kiwi Oil, Laurel Oil, Lanolin and Lanolin derivatives, Lard, Lauric/Palmitic/Oleic Triglyceride, Lavender Oil, Lemongrass Oil, Lemon Oil, Lesquerella Oil, Lovage Oil, Macadamia Nut Oil, Maleated Soybean Oil, Mandarin Orange Oil, Meadowfoarn Seed Oil, Menhaden Oil, Mink Oil, Moringa Oil, Mortierella Oil, Musk Rose Oil, Neatsfoot Oil, Nutmeg Oil, Oleic/Linoleic Triglyceride, Oleic/Paimitic/Lauric/Myristic/Linoleic Triglyceride, Oleostearine, Olibanurn, Olive Husk Oil, Olive Oil, Ornental Lipids, Orange Flower Oil, Orange Oil, Orange Roughy Oil, Palm Kernel Oil, Palm Oil, Peach Kernel Oil, Peanut Oil, Pengawar Djambi Oil, Peppermint Oil, Pentadesma Butter, Phospholipids, Pistachio Nut Oil, Pine Oil, Placental Lipids, Rapeseed Oil, Rice Bran Oil, Rose Hips Oil, Rosemary Oil, Rose Oil, Rue Oil, Safflower Oil, Sage Oil, Sandalwood Oil, Sassafras Oil, Sesame Oil, Shark Liver Oil, Shea Butter, Soybean Oil, Spearmint Oil, Sphingolipids, Sunflower Seed Oil, Sweet Almond Oil, Sweet Marjoram Oil, Sweet Violet Oil, Tall Oil, Tallow, Tea Tree Oil, Thyme Oil, Tribehenin, Tricaprin, Tricaprylin, Triheptanoin, Trihydroxymethoxystearin, Trihydroxystearin, Triisononanoin, Triisostearin, Trilaurin, Trilinolein, Trilinolenin, Trimyristin, Trioctanoin, Triolein, Tripalmitin, Trisebacin, Tristearin, Triundecanoin, Walnut Oil, Wheat Bran Lipids, Wheat Germ Oil, Wild Mint Oil, Yarrow Oil, Ylang Ylang Oil, Zadoary Oil, C10 Fatty Acids: Arachidic Acid, Behenic Acid, Capric Acid, Caproic Acid, Caprylic Acid, Coconut Acid, Corn Acid, Cottonseed Acid, Hydrogenated Coconut Acid, Hydrogenated Menhaden Acid, Hydrogenated Tallow Acid, Hydroxystearic Acid, Isostearic Acid, Lauric Acid, Linoleic Acid, Linolenic Acid, Myristic Acid, Oleic Acid, Palmitic Acid, Palm Kernel Acid, Pelargonic Acid, Ricinoleic Acid, Soy Acid, Stearic Acid, Tallow Acid, Undecanoic Acid, Undecylenic Acid, Wheat Germ Acid, and the like, as well as mixtures thereof. Specific optional lotion conditioning agents found useful in the present invention include panthenol, glycerine, and chamomile oil which are described in detail hereinbelow.

Other optional components known or otherwise effective for use in lotion compositions may also include sterols, phytosterols, and sterol derivatives that act in combination with the natural fats/oils to provide natural skin barrier enhancement and skin barrier recovery. Sterols and sterol derivatives that can be used in the compositions of the invention include, but are not limited to: P-sterols having a tail on the 17 position and having no polar groups for example, cholesterol, sitosterol, stigmasterol, and ergosterol, as well as, C 10-C 30 cholesterol/lanosterol esters, cholecalciferol, cholesteryl hydroxystearate, cholesteryl isostearate, cholesteryl stearate, 7-dehydrocholesterol, dihydrocholesterol, dihydrocholesteryl octyldecanoate, dihydrolanosterol, dihydrolanosteryl octyldecanoate, ergocalciferol, tall oil sterol, soy sterol acetate, lanasterol, soy sterol, avocado sterols, “AVOCADIN” (trade name of Croda Ltd of Parsippany, N.J.), sterol esters and similar compounds, as well as mixtures thereof. A suggested commercially available example of phytosterol is GENEROL 122 N PRL refined soy sterol from Cognis Corporation of Cincinnati, Ohio.

Panthenol: Where included, panthenol typically comprises from about 0.001% to about 10%, preferably from about 0.005% to about 5%, more preferably from about 0.05% to about 1% by weight of the lotion composition. The optional panthenol body conditioning agent provides for skin emolliency benefits that can leave the body feeling smooth, soothing, and soft during and after interaction of the body tissues with the body treatment agents. The lotion compositions of the present invention can include an individual panthenol compound or a mixture of panthenol compounds.

Nonlimiting examples of panthenol include those panthenol compounds which are alcohol or ester derivatives of pantothenic acid. Pantothenic acid is a member of the B complex family and is often referred to as Vitamin B₃. Like pantothenic acid, the panthenol alcohol derivatives of this acid can exist as stereoisomers, for example, the D(+) form, the L(−) form, the racemate, and mixtures of the D(+) and L(−) forms. Specific examples of panthenol include, but are not limited to, D-panthenol (a.k.a. dexpanthenol), and dl-panthenol. Panthenol is more fully described in The Merck Index, Eleventh Edition, entry 2924, p. 464 (1989), which description is incorporated herein by reference. Examples of commercially available panthenol include D-panthenol which is available from Roche Vitamins Incorporation (Nutley, N.J.), a subsidiary of F. Hoffman LaRoche, Ltd.

Glycerine: Where included, the lotion compositions comprise the preferred optional glycerine body conditioning agent at concentrations ranging from about 0.01% to about 10%, preferably from about 0.02% to about 5%, more preferably from about 0.05% to about 2% by weight of the lotion composition. The optional glycerine body conditioning agent also provides for skin emolliency benefits such as smooth, soothing, and soft feeling body, as well as being a dispersing agent for the niacinamide body treatment agent.

Glycerine is a C3 monohydric alcohol that is also referred to as glycerol and 1,2,3-propanetriol. Glycerine derivatives are also suitable for use as an optional skin conditioning agent herein wherein such derivatives include polyglycerols having from about 2 to about 16 repeating glycerol moieties. A specific example of a suitable glycerine skin conditioning agent is Glycerine, USP Kosher® which is commercially available from the Procter & Gamble Company located in Cincinnati, Ohio.

Chamomile: The lotion compositions comprise the preferred optional chamomile oil at concentrations ranging from about 0.0001% to about 10%, preferably from about 0.001% to about 5%, more preferably from about 0.005% to about 2% by weight of the lotion composition. The optional chamomile oil skin conditioning agent also provides for skin benefits such as soothing. Chamomile oil is commonly prepared as an oil extract of chamomile flowers. An example of a commercially available chamomile oil include Phytoconcentrol Chamomile which is available from Dragoco Incorporation (Totowa, N.J.).

Silk Protein or Silk Amino Acids or Silk Peptides: Silk protein is composed of silk fiber and sericin. The silk protein is produced by species of the phylum Arthropoda, classes Insecta and Arachnida. Common species include the silkworm and spider. Sericin acts as a protective envelope around the fibroin thread as it is spun and can be easily separated from silk protein by chemical processes such as hydrolysis. Sericin and/or silk amino acids and/or silk peptides are amenable to binding to the skin and hair, forming a resistant, moisturizing, and protective film on the skin/hair. The optional silk also provides for body benefits such as soothing, moisturizing, and conditioning. Another example of a silk derivative for use in the lotion composition of the present invention is a fine powder of silk fibroin in nonfibrous or particulate form, as disclosed in U.S. Pat. No. 4,233,212 to Otoi et al., incorporated herein by reference in its entirety. The lotion compositions comprise the preferred optional silk protein or silk amino acids, or mixtures thereof at concentrations ranging from about 0.0001% to about 25%, preferably from about 0.0005% to about 15%, more preferably from about 0.001% to about 10% by weight of the lotion composition. Silk protein or silk derived amino acids or peptides is commonly prepared as a powder or as part of an aqueous solution. Silk proteins generally have an average molecular weight of >8000 and can have molecular weights of >500.000. Silk amino acid or silk peptide solutions typically have lower molecular weights which may typically be described as between 40 and 400. A commercially available silk protein is available from Croda, Inc., of Parsippany, N.J., and is sold under the trade name CROSILK® LIQUID (silk amino acids), CROSILK® 10,000 (hydrolyzed silk), CROSILK® POWDER (powdered silk), and CROSILKQUAT (cocodimonium hydroxypropyl silk amino acid). Another example of a commercially available silk protein is sericin, available from Pentapharm, LTD, a division of Kordia, by, of the Netherlands. Further details of such silk protein mixtures can be found in U.S. Pat. No. 4,906,460, to Kim, et al., assigned to Sorenco, which is herein incorporated by reference in its entirety. Other sources of silk include Collaborative Laboratories Silkall 100®, Silkpro®, and SilPRO® Silk Essence.

### Branched Chain Amino Acids and Enzyme Activator:

The lotion compositions can comprise the preferred optional branched chain amino acids and enzyme activator, or mixtures thereof at a at concentrations ranging from about 0.0001% to about 40%, preferably from about 0.001% to about 30%, more preferably from about 0.001% to about 10% by weight of the lotion composition. Branched chain amino acids are selected from the group leucine, isoleucine, and valine or their derivatives or their metabolites. The enzyme activators are selected from the group consisting of octanoic acid and its derivatives, hexanoic acid and its derivatives, alpha ketoisocaproic acid and its derivatives, thiamin diphosphate and its derivatives and related materials capable of activating the enzyme, branched-chain keto acid dehydrogenase. The amino acid derivatives or metabolites are selected from the group consisting of: nor-leucine, nor-valine, L-alloisoleucine, L-threo-isoleucine, D, L, or DL-leucine-containing di- and tri-peptides, isovaleryl-CoA, isovalerylcamitine, alpha-methylbutyrylglycine, alpha-methylbutyric acid, and related amino acids and metabolites as described in U.S. Pat. No. 6,149,924 issued to Harbhajan Paul on Nov. 21, 2000. Leucine, isoleucine, and valine can be obtained under the tradename AEC Leucine, AEC Isoleucine, and AEC Valine, respectfully, from A&E Connock (Hampshire, United Kingdom). Octanoic acid and hexanoic acid can be obtained under the tradename AEC Caprylic Acid and AEC Caproic Acid, respectfully, from A&E Connock (Hampshire, United Kingdom).

### Phytosteroids

Phytosteroids represent materials that are extracted from plants. Representative ingredients can include steroidal and non-steroidal structures both possessing steroid-like biological activity. Examples of steroidal materials include vegetable oil derived steroids, i.e., sitosterol, stigmasterol, and campesterol. Non-steroidal structures include isoflavones, flavones, and coumestans. Isoflavones, which include genestein, daidzein, formononetin, and equol have been identified as useful treatments for symptoms associated with menopause and perimenopause (U.S. Pat. No. 5,498,631 to GORBACH Mar. 12, 1996), depression and dementia (U.S. Pat. No. 5,733,926 to GORBACH Mar. 31, 1998, U.S. Pat. No. 6,083,526 to GORBACH, Jul. 4, 2000), skin wrinkling (U.S. Pat. No. 6,060,070 to GORBACH May 9, 2000), and cancer (WO 2004022023 to NOVOGEN). The principal route of delivery is through pills, food, suppositories, cosmetic formulas or, restricted to sun exposed areas of the body (i.e., to treat and prevent solar actinic damage as described in WO 2004026274 to NOVOGEN and WIDYARIM S. ET AL.: ‘Isoflavonoid compounds from red clover (Trifolium pratense) protect from inflammation and immune suppression induced by UV radiation’ PHOTOCHEMISTRY AND PHOTOBIOLOGY vol. 74, no. 3, 2001, pages 465-470.). Since these isoflavones are known to have multiple effects in the body, and to avoid any undesirable side effects, it is critical that the isoflavones be targeted to the tissue/organ of interest at the appropriate dosage and be delivered in a way that will ensure compliance, i.e., what might be appropriate for oral administration will not be the same for topical administration, and what might be appropriate as a cosmetic composition for the face and arms will not be acceptable in the vulvar area, and in particular for an absorbent article. Furthermore, although there is disclosure for delivering isolaflavones through the skin using a skin patch composition (U.S. Pat. No. 6,083,526 to GORBACH, Jul. 4, 2000), it is common knowledge that the adhesives used in skin patches can be irritating to the vulvar skin and be uncomfortable to wear, thus compromising user compliance. There is also disclosure of the use of a combination of isoflavones and microorganisms in mature women to treat symptoms associated with older women (U.S. Pat. No. 6,716,424 to OTSUKA PHARM CO LTD Apr. 6, 2004) or a combination of isoflavones and steroids (US 2004131579A to AVON). By modifying the natural ecoflora found in this heavily colonized part of the body, this treatment would pose significant health risk when applied to the vulvar area. Steroids are well known to carry a systemic health risk.

Thus there is a need to target the delivery of a safe composition of isoflavones to the target skin of interest, there is need to formulate the isoflavones in a carrier to deliver the isoflavones to the target of interest, there is a need to use a device to deliver the isoflavones to the vulvar area, there is a need for the carrier containing isoflavones to be compatible with an absorbent article that is normally used by females and whose carrier containing isoflavones can be transferred to the body the wearer.

The lotion composition can include 0.001% to about 40% of isoflavones, preferably 0.001% to about 4%, more preferably 0.01% to about 0.5% isoflavones. The isoflavones can be selected from the group consisting of soy isoflavones, clover isoflavones, genestein, daidzein, formononetin, biochanin A, S-equol, R-equol or a mimetic plant extract. By mimetic plant extract is meant, in the context of the application, any plant extract capable of mimicking the action of the isoflavones identified.

### Plant Extract:

The lotion compositions can comprise the preferred optional plant extract at concentrations ranging from about 0.0001% to about 40%, preferably from about 0.001% to about 20%, more preferably from about 0.001% to about 5% by weight of the lotion composition. The plant extract is obtainable by grinding a whole plant or one or more of plant parts (e.g., leaves, bark, roots, branches, etc.), either dried or undried, and extracting with a solvent at ambient temperature or under heating or by means of an extractor, e.g., a Soxhlet's extractor. Extracted plant (or botanical) actives can include any water-soluble or oil-soluble active extracted from a particular plant. Botanical extracts are generally available from commercial suppliers as part of a composition that also contains an extracting solvent. Amounts of the botanical extract in the compositions of the present invention in terms of active component may range from about 0.000001% (by total weight of the composition) to about 10% (by total weight of the composition). In addition, the botanical extracted actives can be supplied as a powder. Any plant extract can be used with no particular restriction as long as it has no apparent adverse effect on the absorbent article's physical integrity (i.e., dissolves adhesive glues) or substantially no decomposing action on the absorbent core. Examples of botanical extracts include, but are not limited to absinthe, Agnus castus, alga (e.g., bladder wrack (Fucus vesiculosis)), aloe vera, apple, Areca catechu, arnica, basil leaves, bell pepper, Beta vulgaris (beet), blackberry, black cohosh, black currant fruit, black tea, blueberry, borage seed calendula, carrot root, centella asiatica, chamomile, Chinese tea, chokeberry, Chuck Berry, citron, coffee seed, comfrey, cornflower, crowberry, dandelion root, date palm fruit, dong quad, echinacea, evening primrose, eucalyptus, fenugreek, fennel fruit, field horsetail, gingko, gooseberry, grapefruit fruit, grape seed extract, green tea, hamamelis, hawthorn berries, hiba arborvitae, horse chestnut, Hypericum pefforatum, ivy (Herdera helix), lavender, lemon balm, licorice, magnolia, mimosa, oat, mung bean, oolong tea, oregano, pansey, peach leaf, persimmon, philodendron, Rhododendron, rosemary, sage, St. John's wort, scullcap, seabuckthorn, strawberry, sweet pea, thyme, tomato, Turkish oregano, vanilla fruit, yucca glauca, white nettle, whortleberry, willow herb, and witch hazel. Another group of botanical extracts include perfume or essential oils.

Examples of essential oils that can be incorporated in the lotion composition include: Anise Oil, Balm Mint Oil, Basil Oil, Bee Balm Oil, Bergamot Oil, Birch Oil, Bitter Almond Oil, Bitter Orange Oil, Calendula Oil, California Nutmeg Oil, Caraway Oil, Cardamom Oil, Chamomile Oil, Cinnamon Oil, Clary Oil, Cloveleaf Oil, Clove Oil, Coriander Oil, Cypress Oil, Eucalyptus Oil, Fennel Oil, Gardenia Oil, Geranium Oil, Ginger Oil, Grapefruit Oil, Hops Oil, Hyptis Oil, Indigo Bush Oil, Jasmine Oil, Juniper Oil, Kiwi Oil, Laurel Oil, Lavender Oil, Lemongrass Oil, Lemon Oil, Linden Oil, Lovage Oil, Mandarin Orange Oil, Matricaria Oil, Musk Rose Oil, Nutmeg Oil, Olibanum, Orange Flower Oil, Orange Oil, Patchouli Oil, Pennyroyal Oil, Peppermint Oil, Pine Oil, Pine Tar Oil, Rose Hips Oil, Rosemary Oil, Rose Oil, Rue Oil, Sage Oil, Sambucus Oil, Sandalwood Oil, Sassafras Oil, Silver Fir Oil, Spearmint Oil, Sweet Marjoram Oil, Sweet Violet Oil, Tar Oil, Tea Tree Oil, Thyme Oil, Wild Mint Oil, Yarrow Oil, Ylang Ylang Oil, and the like, as well as mixtures thereof. Perfume oils are natural odorous substances that are generally extracted from blossoms (lilly, lavender, rose, jasmine, neroli, ylang-ylang), from stems and leaves (geranium, patchouli, petitgrain), from fruits (anis, coriander, caraway, juniper), from cortex (bergamot, lemon, orange), from roots (macis, angelic, celery, cardamon, costus, iris, calmus), from wood (pine, sandelwood, guajak, cedar, rose), from herbs and grass (tarragon, lemongrass, sage, thyme), from needles and branches (spruce, fir, pine, mountain pine), from resins and balms (galbanum, elemi, benzoin, myrrh, olibanum, opoponax).

II. Carrier:

The lotion compositions of the present invention comprise a carrier for the body treatment agents. The carrier can be included in the compositions as an individual carrier or a combination of carrier ingredients, provided that the total carrier concentration is sufficient to provide transfer and/or migration of the body treatment agents onto the body. The carrier can be a liquid, solid, or semisolid carrier material, or a combination of these materials, provided that the resultant carrier forms a homogenous mixture or solution at selected processing temperatures for the resultant carrier system and at processing temperatures for combining the carrier with the skin treatment agents in formulating the lotion compositions herein. Processing temperatures for the carrier system typically range from about 60° C. to about 90° C., more typically from about 70° C. to about 85° C., even more typically from about 70° C. to about 80° C.

The lotion compositions of the present invention typically comprise the carrier at a total carrier concentration ranging from about 60% to about 99.9%, preferably from about 70% to about 98%, more preferably from about 80% to about 97% by weight of the lotion composition. Suitable carrier compounds include petroleum-based hydrocarbons having from about 4 to about 32 carbon atoms, fatty alcohols having from about 12 to about 24 carbon atoms, polysiloxane compounds, fatty acid esters, alkyl ethoxylates, lower alcohols having from about 1 to about 6 carbon atoms, low molecular weight glycols and polyols, fatty alcohol ethers having from about 12 to about 28 carbon atoms in their fatty chain, lanolin and its derivatives, glyceride and its derivatives including acetoglycerides and ethoxylated glycerides of C12-C28 fatty acids, and mixtures thereof. Alternatively or in combination with, the carrier may also be composed of polysiloxane compounds non-limiting examples include dimethicones (1-100,000,000 centistoke), cyclomethicones, alkylated silicones (hair conditioning agents), silicone gums, silicone gels, silicone waxes, copolymers of silicone (vinyl dimethicone polymers, phenyl vinyl dimethicone polymers, alkylated silicone polymers, polyethylene oxide/silicone copolymers, polyethylene oxide/alkyl silicone copolymers), and mixtures thereof.

Nonlimiting examples of suitable petroleum-based hydrocarbons having from about 4 to about 32 carbon atoms include mineral oil, petrolatum, isoparaffins, various other branched chained hydrocarbons, and combinations thereof. Mineral oil is also known as “liquid petrolatum”, and usually refers to less viscous mixtures of hydrocarbons having from about 16 to about 20 carbon atoms. Petrolatum is also known as “mineral wax”, “petroleum jelly”, and “mineral jelly”, and usually refers to more viscous mixtures of hydrocarbons having from about 16 to about 32 carbon atoms. An example of commercially available petrolatum include petrolatum sold as Protopet® 1 S which is available from the Witco Corporation located in Greenwich, Conn.

Nonlimiting examples of suitable fatty alcohols having from about 12 to about 24 carbon atoms include saturated, unsubstituted, monohydric alcohols or combinations thereof, which have a melting point less than about 110° C., preferably from about 45° C. to about 110° C. Specific examples of fatty alcohol carriers for use in the lotion compositions of the present invention include, but are not limited to, cetyl alcohol, stearyl alcohol, cetearyl alcohol, behenyl alcohol, arachidyl alcohol, lignocaryl alcohol, and combinations thereof. Examples of commercially available cetearyl alcohol is Stenol 1822 and behenyl alcohol is Lanette 22, both of which are available from the Cognis Corporation located in Cincinnati, Ohio.

Nonlimiting examples of suitable fatty acid esters include those fatty acid esters derived from a mixture of C₁₂-C₂₈ fatty acids and short chain (C₁-C₈, preferably C₁-C₃) monohydric alcohols preferably from a mixture of C₁₆-C₂₄ saturated fatty acids and short chain (C₁-C₈, preferably C₁-C₃) monohydric alcohols. Representative examples of such esters include methyl palmitate, methyl stearate, isopropyl laurate, isopropyl myristate, isopropyl palmitate, ethylhexyl palmitate, and mixtures thereof. Suitable fatty acid esters can also be derived from esters of longer chain fatty alcohols (C₁₂-C₂₈, preferably C₁₂-C₁₆) and shorter chain fatty acids such as lactic acid, specific examples of which include lauryl lactate and cetyl lactate.

Nonlimiting examples of suitable alkyl ethoxylates include C₁₂-C₂₂ fatty alcohol ethoxylates having an average degree of ethoxylation of from about 2 to about 30. Nonlimiting examples of suitable lower alcohols having from about 1 to about 6 carbon atoms include ethanol, isopropanol, butanediol, 1,2,4-butanetriol, 1,2 hexanediol, ether propanol, and mixtures thereof. Nonlimiting examples of suitable low molecular weight glycols and polyols include ethylene glycol, polyethylene glycol (e.g., Molecular Weight 200-600 g/mole), butylene glycol, propylene glycol, polypropylene glycol (e.g., Molecular Weight 425-2025 g/mole) and mixtures thereof. A more detailed description of carrier ingredients including suitable hydrocarbons, polysiloxane compounds, and fatty alcohol ethoxylates can be found in U.S. Pat. No. 5,643,588, issued Jul. 1, 1997 to Roe et al. entitled “Diaper Having A Lotioned Topsheet”.

In one embodiment, the carrier comprises a combination of one or more petroleum-based hydrocarbons and one or more fatty alcohols described hereinabove. When one or more petroleum-based hydrocarbons having from about 4 to about 32 carbon atoms are used in combination with one or more fatty alcohols having from about 12 to about 22 carbon atoms, the petroleum-based hydrocarbons are included at total concentrations ranging from about 20% to about 99%, preferably from about 30% to about 85%, more preferably from about 40% to about 80% by weight of the lotion composition; wherein the fatty alcohols are included at total concentrations ranging from about 0.2% to about 65%, preferably from about 1% to about 50%, more preferably from about 2% to about 40% by weight of the lotion composition.

It is believed that a petroleum-based carrier system comprising C₄-C₃₂ hydrocarbons, C₁₂-C₂₂ fatty alcohols, and fumed silica provides a homogeneous mixture of the carrier, skin treatment agents, and any optional ingredients wherein this homogeneous mixture ensures sufficient contact between the skin and skin treatment agents to result in effective prevention and treatment of skin disorders. The fumed silica suitable for inclusion in the preferred petroleum-based carrier system, or with any other carrier described herein, includes colloidal pyrogenic silica pigments which are sold under the Cab-O-Sil® tradename, and which are commercially available from the Cabot Corporation located in Tuscola, Ill. These colloidal pyrogenic silica pigments are submicroscopic particulated pyrogenic silica pigments having mean particle sizes ranging from about 0.1 microns to about 100 microns. Specific examples of commercially available Cab-O-Sil® silica pigments include Cab-O-Sil® TS-720 (a polydimethylsiloxane treated fumed silica), Cab-O-Sil® TS-530 (a trimethyl silanized fumed silica), and Cab-O-Sil® TS-610 (a dimethyldisilanized fumed silica). The fumed silica provides the lotion compositions with desired viscosity or thickening properties, and is typically included at concentrations ranging from about 0.01% to about 15%, preferably from about 0.1% to about 10%, more preferably from about 1% to about 5% by weight of the lotion composition.

The fumed silica can be used alone or in combination with other optional viscosity, thickening, or immobilizing agents such as talc, bentonites including treated bentonites, hectorites including treated hectorites, calcium silicates including treated calcium silicates, magnesium silicates, magnesium aluminum silicates, zinc stearates, sorbitol, colloidal silicone dioxides, spermaceti, carnuba wax, beeswax, candelilla wax, paraffin wax, microcrystalline wax, castrol wax, ceresin, esparto, ouricuri, rezowax, polyethylene wax, C₁₂-C₂₄ fatty acids, polyhydroxy fatty acid esters, polyhydroxy fatty acid amides, polymethacrylate polymers, polymethacrylate and styrene copolymers, and combinations thereof. These other optional viscosity modifying or thickening or immobilizing agents are also included at total concentrations ranging from about 0.01% to about 15%, preferably from about 0.1% to about 10%, more preferably from about 0.1% to about 10% by weight of the lotion composition. A nonlimiting specific example of another suitable viscosity or thickening agent include bentonite sold as Bentone® 38 which is available from the Rheox Incorporation.

Other non-limiting examples of suitable immobilizing agents include natural or synthetic waxes. As used herein, the term ‘wax’ refers to oil soluble materials that have a waxy constituency and have a melting point or range of above ambient temperature, in particular above 25° C. Waxes are materials that have a solid to semi-solid (creamy) consistency, crystalline or not, being of relative low viscosity a little above their liquefying point. Waxes can be composed of one or more components, synthetic as well as natural, and can in principle be composed of or comprise any oil soluble material having a waxy constituency, including mixtures thereof. Suitable waxes which can be incorporated into the lotion include: beeswax, candelilla wax, carnauba wax, ceresine wax, cocoa butter, cork wax, esparto gras wax, fruit-derived waxes, such as bayberry wax, orange wax, lemon wax, grapefruit wax and bayberry wax, and the like, Guaruma wax, hydrogenated jojoba waxes, hydrogenated microcrystalline wax, hydrogenated rice bran wax, japan wax, jojoba wax, lanolin wax, mineral waxes such as ceresine ouricury, and ozokerite waxes, mink wax, montan wax, motan acid wax, motan wax, paraffin, PEG-6 beeswax, PEG-8 beeswax, polyalkylene and polyethyleneglycol waxes, rezowax, rice bran wax, rice shoot wax, Sasol waxes, shellac wax, spent grain wax, spermateci, sugar cane wax, sunflower wax, synthetic beeswax, synthetic candelilla wax, synthetic carnuba wax, synthetic japan wax, synthetic jojoba wax, wool wax, waxes based on chlorinated napthalenes such as ‘Halowax’,

It is preferable that the carrier be hydrophobic. Further, it is preferable that the lotion composition of the present invention comprise no surfactant. Therefore, in a preferred embodiment of the present invention the lotion has a level of hydrophobicity at least as great as that of the topsheet, and the hydrophobicity of the lotion is primarily due to the lack of a surfactant component. If, under some condition, there is a need to raise the wettability of the hydrophobic carrier one may optionally add a wetting agent such as polyoxyethylene alkyl ethers, alkyl ethoxylates, alkylethoxylated amines, polyethylene glycol esters, and/or sorbitan fatty acid esters generally having a low degree of ethoxylation and HLB values below about 7. Suitable additives will be miscible with the carrier so as to form a homogenous mixture. Because of possible skin sensitivity of those using the catamenial device of the present invention, these wetting agents should also be relatively mild and non-irritating to the skin. Typically, these wetting agents are nonionic to be not only non-irritating to the skin, but also to avoid other undesirable effects on any underlying tissue laminate structure, e.g., reductions in tensile strength. Suitable wetting agents will typically have HLB values below 10, preferably below 9, more preferably below 8, and even more preferably below 7.

Non-limiting specific examples of a suitable wetting agents includes nonyl phenol or or polyoxyethylene nonyl phenyl ether (20 of ethoxylation; HLB of 5.7), octyl phenol or polyoxyethylene octyl phenyl ether (10 of ethoxylation; HLB of 3.5), stearyl alcohol or polyoxyethylene stearyl ether (20 of ethoxylation; HLB of 4.9), stearyl amine or polyoxyethylene stearyl amine (20 of ethoxylation; HLB of 4.9), polyethylene glycol 200 dilaurate (HLB 5.9), polyethylene glycol 200 distearate (HLB 4.8), sorbitan monostearate (‘Span 60’ having HLB 4.7), sorbitan tristearate (‘Span 65’ having HLB 2.1), sorbitan monooleate (‘Span 80’ having HLB 4.3), sorbitan trioleate (‘Span 85’ having HLB 1.8), each of which are available form Cell Chemical Company (Inchon, Korea) or Uniqema (New Castle, Del., USA).

Other not-limiting examples of nonionic surfactants for use in lotion compositions of the present invention include alkylglycosides; alkylglycoside ethers as described in U.S. Pat. No. 4,011,389 (Langdon, et al), issued Mar. 8, 1977; alkylpolyethoxylated esters, ethoxylated sorbitan mono-, di- and/or tri-esters of C₁₂-C₁₈ fatty acids having an average degree of ethoxylation of from about 2 to about 20, preferably from about 2 to about 10, and the condensation products of aliphatic alcohols with from about 1 to about 54 moles of ethylene oxide. The alkyl chain of the aliphatic alcohol is typically in a straight chain (linear) configuration and contains from about 8 to about 22 carbon atoms. Particularly preferred are the condensation products of alcohols having an alkyl group containing from about 11 to about 22 carbon atoms with from about 2 to about 30 moles of ethylene oxide per mole of alcohol.

The amount of wetting agent required to increase the wettability of the lotion composition to a desired level will depend upon its HLB value and HLB level of the carrier used, and like factors. The lotion composition can comprise from about 1 to about 50% of the wetting agent when needed to increase the wettability properties of the composition. Preferably, the lotion composition comprises from about 0.1% to about 25%, or from about 1% to about 15%, or from about 1% to about 10%, or from about 5% to about 20% of the wetting agent when needed to increase wettability.

Other ingredients may be incorporated into the composition such as viscosity modifiers, perfumes, disinfectant antibacterial actives, pharmaceutical actives, film formers, deodorants, opacifiers, astringents, solvents and the like. In addition, stabilizers can be added to enhance the shelf life of the lotion composition such as cellulose derivatives, proteins and lecithin. All of these materials are well known in the art as additives for such formulations and can be employed in appropriate amounts in the lotion compositions of the present invention.

III. Absorbent Article

The lotion compositions of the present invention are preferably transferred to the body from application of the compositions onto a catamenial device. These products may comprise a topsheet, a backsheet, and an absorbent core positioned between the topsheet and backsheet; each component having a body- or wearer-contacting surface and a garment surface. The terms “body-contacting surface” and “wearer-contacting surface” are used interchangeably herein and refer to one or more surfaces of any article component that is intended to be worn or positioned toward or adjacent the body of the wearer/user for contact between the wearer/user and the article's surface at some time during the use period. The term “garment surface” as used herein refers to the outer or exterior surface of any article component that is intended to be worn or positioned adjacent a wearer's undergarments, or in the case of an absorbent article which is not worn by the user, the garment surface is typically positioned adjacent a user's hand or other implement assisting in the use of the absorbent article. As used herein, the term “wearer” and “user” are used interchangeably as the present invention contemplates absorbent articles which may not be intended to be worn, but rather used to absorb bodily exudates while transferring the lotion compositions of the present invention.

A. Topsheet: The absorbent article may comprise any known or otherwise effective topsheet, such as one which is compliant, soft feeling, and non-irritating to the wearer's body. Suitable topsheet materials include a liquid pervious material that is oriented towards and contacts the body of the wearer permitting bodily discharges to rapidly penetrate through it without allowing fluid to flow back through the topsheet to the skin of the wearer. The topsheet, while being capable of allowing rapid transfer of fluid through it, also provides for the transfer or migration of the lotion composition onto an external or internal portion of a wearer's body. A suitable topsheet can be made of various materials such as woven and nonwoven materials; apertured film materials including apertured formed thermoplastic films, apertured plastic films, and fiber-entangled apertured films; hydro-formed thermoplastic films; porous foams; reticulated foams; reticulated thermoplastic films; thermoplastic scrims; or combinations thereof, as is well known in the art of making catamenial products such as sanitary napkins, pantiliners, incontinence pads, and the like.

When the topsheet comprises a nonwoven fibrous material in the form of a nonwoven web, the nonwoven web may be produced by any known procedure for making nonwoven webs, nonlimiting examples of which include spunbonding, carding, wet-laid, air-laid, meltblown, needle-punching, mechanical entangling, thermo-mechanical entangling, and hydroentangling.

B. Backsheet: The catamenial device of the present invention also comprises a backsheet. The backsheet can be any known or otherwise effective backsheet material, provided that the backsheet prevents external leakage of exudates absorbed and contained in the catamenial device. Flexible materials suitable for use as the backsheet include, but are not limited to, woven and nonwoven materials, laminated tissue, polymeric films such as thermoplastic films of polyethylene and/or polypropylene, composite materials such as a film-coated nonwoven material, or combinations thereof, as is well known in the art of making catamenial products such as sanitary napkins, pantiliners, incontinence pads, and the like.

C. Absorbent Core: The catamenial device also comprises an absorbent. The absorbent core is typically positioned between the topsheet and the backsheet. As used herein, the term “absorbent core” refers to a material or combination of materials suitable for absorbing, distributing, and storing aqueous fluids such as urine, blood, menses, and water found in body exudates. The size and shape of the absorbent core can be altered to meet absorbent capacity requirements, and to provide comfort to the wearer/user. The absorbent core suitable for use in the present invention can be any liquid-absorbent material known in the art for use in absorbent articles, provided that the liquid-absorbent material can be configured or constructed to meet absorbent capacity requirements. Nonlimiting examples of liquid-absorbent materials suitable for use as the absorbent core include comminuted wood pulp which is generally referred to as airfelt; creped cellulose wadding; absorbent gelling materials including superabsorbent polymers such as hydrogel-forming polymeric gelling agents; chemically stiffened, modified, or cross-linked cellulose fibers; meltblown polymers including coform; synthetic fibers including crimped polyester fibers; tissue including tissue wraps and tissue laminates; capillary channel fibers; absorbent foams; absorbent sponges; synthetic staple fibers; peat moss; or any equivalent material; or combinations thereof, as is well known in the art of making catamenial products such as sanitary napkins, pantiliners, incontinence pads, and the like.

IV. Methods of Treating the Body:

The present invention also relates to methods of treating the body with the lotion compositions described herein. Generally, a safe and effective amount of the lotion composition is applied to an absorbent article described herein wherein such safe and effective amounts include applying from about 0.0015 mg/cm² (0.01 mg/in²) to about 100.5 mg/cm² (100 mg/in²), preferably from about 0.003 mg/cm² (0.02 mg/in²) to about 12.4 mg/cm² (80 mg/in²), more preferably from about 0.02 mg/cm² (0.015 mg/in²) to about 7.75 mg/cm² (50 mg/in²), of the lotion composition to the absorbent article.

Typically, a safe and effective amount of the lotion compositions of the present invention is applied to an absorbent article such that at least about 0.00015 mg/cm² (0.001 mg/in²) to about 15.5 mg/cm² (100 mg/in²), preferably from about 0.0006 mg/cm² (0.004 mg/in²) to about 11 mg/cm² (72 mg/in²), more preferably from about 0.005 mg/cm² (0.03 mg/in²) to about 6.2 mg/cm² (40 mg/in²), of the composition is transferred to the body during a single use of an absorbent article which is typically about a three hour period. Absorbent articles are generally changed every three to six hours during the day and once for overnight protection, resulting in at least a safe and effective amount of from about 0.00045 mg/cm² (0.003 mg/in²) to about 124 mg/cm² (800 mg/in²), preferably from about 0.0018 mg/cm² (0.012 mg/in²) to about 88 mg/cm² (576 mg/in²), more preferably from about 0.015 mg/cm² (0.09 mg/in²) to about 49.6 mg/cm² (320 mg/in²), of the lotion composition being administered within a one day interval (24 hour period). However, the transfer of the lotion compositions of the present invention onto a wearer's body via an absorbent article described herein can occur for one day, several days, weeks, months, or years at appropriate intervals provided that safe and effective amounts of the lotion compositions are administered to deliver the body treatment benefits described herein.

### Process for Applying the Compositions

The lotion compositions of the present invention can be applied to the absorbent articles by any known or otherwise effective technique for distributing a lotion composition onto an absorbent product such as a disposable absorbent article. Nonlimiting examples of methods of applying the lotion compositions onto an absorbent article include spraying, printing (e.g., flexographic printing), coating (e.g., contact slot coating and gravure coating), extrusion, or combinations of these application techniques. The application of the lotion compositions onto an absorbent article facilitates the transfer or migration of the lotion compositions onto the skin for administration and/or deposition of the lotion compositions, resulting in a safe and effective amount of the compositions being applied for improved prevention and reduction of skin disorders. Therefore, the safe and effective amount of the lotion composition that will transfer or migrate to the body will depend on factors such as the type of lotion composition that is applied, the portion of the body contacting surface where the lotion composition is applied, and the type of absorbent article used to administer the lotion composition.

Any suitable method can be used in determining the amount of a lotion composition described herein that is transferred to the body of a wearer during use of an absorbent article containing the composition. An example of specific methods for the calculation of transfer amounts of lotion compositions include Gas Chromatographic and other quantitative analytical procedures that involve the analysis of in vivo skin analog materials. A suitable Gas Chromatographic procedure is more fully described in WO 99/45973, Donald C. Roe et al, published Sep. 16, 1999.

In preparing absorbent articles according to the present invention, the composition may be applied to a topsheet and/or cuffs as liquid or as a semi-liquid wherein the particulate material described herein is dispersed. Typically, the composition is applied to the outer surface or outer surfaces that in use are in contact with the skin of the user.

The particles typically have the same structure and particle sizes when added to the liquid composition as when present on the articles and/or transferred to the skin of the user (e.g. unlike for example lotion crystals that are liquid when applied to the articles and that melt when applied to the skin). The lotion composition is typically applied from a melt thereof to the absorbent article topsheet.

Since the (lotion) composition melts at above-ambient temperatures, it is usually applied as a heated composition to the topsheet or cuffs. Typically, the lotion composition is heated to a temperature in the range from about 40° to about 100° C., preferably from 50° or even from 60° C. to about 100 C. or even to 90° C., prior to being applied to the topsheet or cuffs.

The particles are added to the liquid or semi-liquid carrier component and evenly mixed therewith to ensure the particles are homogeneously dispersed therein and the resulting composition is a homogeneous mixture with the particles dispersed in a homogenous manner. This can then be applied to the topsheet or cuffs.

Typically, the liquid or semi-liquid component and/or the composition is heated to ensure it is liquid or semi-liquid. Then, once the composition has been applied to the topsheet or cuffs, it is allowed to cool and solidify to form solidified coating on the surface of the topsheet or cuffs, wherein the particles typically remain the same structure and/or particle size distribution as prior to application.

Where the substance is applied intermittently, any pattern may be utilized, including, for example, application of small droplets (obtained via, e.g., spraying) discrete dots (obtained via, e.g., gravure printing), alternating stripes that run in the longitudinal or lateral direction of the article, etc. By alternating stripes is meant regions in which the lotion is applied as stripes separated by regions which have no lotion applied. The stripes may have a width from between 0.1 mm to about 50 mm, from between 0.1 to about 30 mm, from between 0.5 mm to about 50 mm, from about 0.5 mm to about 40 mm, from between 2 mm to about 40 mm, from between 2 mm to about 20 mm, from between 2 mm to about 15 mm, or from between 5 mm to about 20 mm. The spacing between the stripes having no lotion applied may have a width from between 0.1 mm to about 100 mm, from about 0.1 mm to about 50 mm, from between 0.1 to about 30 mm, from between 0.5 mm to about 50 mm, from about 0.5 mm to about 40 mm, from between 2 mm to about 40 mm, from between 2 mm to about 20 mm, from between 2 mm to about 15 mm, or from between 5 mm to about 20 mm. The substance can be applied directly to the absorbent article topsheet, or it may be applied to another material or component which is then adhered to the desired portion of the absorbent article (such as a calender roll). The substance can cover any of the following percentages of the surface area of the body-contacting surface of the main body portion, the central absorbent portion, the flexible extensions, or the entire body-contacting surface of the absorbent article topsheet. In those embodiments where the body-contacting surface of the main body portion, comprises discrete, untreated regions, the percent open area of the region of the body-contacting surface can vary widely. (As referred to herein, the “percent open area” of the body-contacting surface is determined by (i) measuring the surface area of the body-contacting surface, (ii) measuring the total surface area of the untreated region(s) of the body-contacting surface and (iii) dividing the measurement in (ii) by the measurement in (i). As used herein, “untreated” means a region of the body-contacting surface having less than about 0.02 g/m2 of substance. In this regard, the percent open area may be from about 1% to about 99%, from about 5% to about 95%, from about 10% to about 90%, from about 15% to about 85%, from about 20% to about 80%, from about 25% to about 75%, from about 30% to about 70%, or from about 35% to about 65%. The percent open area required to achieve the desired substance effect and the desired fluid handling properties of the topsheet will be dictated largely by the characteristics of the substance (in particular the lotion's composition and its relative hydrophobicity/hydrophilicy properties).

V. Method of Manufacture:

The lotion compositions of the present invention may be prepared by any known or otherwise effective technique, suitable for providing a lotion composition comprising the essential skin treatment agents defined herein. In general, the lotion compositions are prepared by first making a carrier system comprising suitable carriers such as petrolatum and behenyl alcohol in combination with a fumed silica thickening agent. Next, a mixture comprising the skin treatment agents and any optional ingredients such as optional skin conditioning agents are added to the carrier system at a melt mix temperature of about 80° C. Although the carrier system, skin treatment agents, and any optional ingredients are typically processed at a temperature of about 80° C., these materials can be processed at temperatures ranging from about 60° C. to about 90° C., preferably from about 70° C. to about 90° C. The resultant lotion composition is subsequently applied to a topsheet component of an absorbent article using a contact applicator such as a Nordsen EP 11-12-02.

The lotion compositions of the present invention are prepared such that the compositions can be applied to an absorbent article to result in safe and effective amounts of the compositions being transferred onto the skin of a wearer of the absorbent article. Therefore, the lotion compositions preferably have a product consistency such that they are relatively immobile and localized on the wearer-contacting surface of the absorbent article at ambient conditions, are readily transferable to the wearer at body temperature, and yet are not completely liquid under extreme storage conditions. In other words, the lotion compositions are solids or semisolids at ambient conditions (about 25° C.) and/or body temperature (about 37° C.) so that the compositions are easily transferred onto the skin by way of normal contact, wearer motion, and/or body heat. The consistency of the lotion compositions can be measured according to ASTM D5 test method which involves the use of a penetrometer to measure consistency. Typically, the lotion compositions of the present invention have a consistency of from about 10 to about 300, preferably from about 20 to about 250, more preferably from about 30 to about 200, as measured at 40° C. according to the test procedure outlined in ASTM D5 test method.

The solid or semisolid consistency of the lotion compositions provide for relatively low levels of the compositions to be applied to the absorbent articles to impart the desired lotion benefits. By “semisolid” is meant that the compositions have a rheology typical of pseudoplastic or plastic liquids such that the compositions remain relatively stationary in a desired location on the absorbent article, and do not have a tendency to flow or migrate to undesired locations of the article. The solid lotion compositions of the present invention likewise can remain in a particular location and not flow or migrate to undesired locations of the article. These solid and semisolid lotion compositions have viscosities high enough to keep the compositions localized on an intended location of the article, but not so high as to impede transfer to the wearer's skin. Typically, final products of solid and semisolid lotion compositions have viscosities ranging from about 1.0×10⁶ centipoise to about 1.0×10¹⁰ centipoise under shear stress conditions of about 3×10³ dynes/cm² at 40° C. (the shear stress applied to the compositions while the absorbent article is in storage or transported at temperature conditions of about 40° C.).

However, the solid and semisolid lotion compositions can be made flowable for transfer or migration of the compositions onto the skin by applying shear stress that results in deformation of the compositions. The shear stress applied at least once during wear of the absorbent article under temperature conditions of about 40° C. is typically at about 1.0×10⁶ dynes/cm², and this shear stress can result in the lotion compositions having a viscosity of from about 1.0×10¹ centipoise to about 1.0×10⁵ centipoise. It is believed that the lotion compositions achieve the lower viscosity values under applied shear stress due to the fact that, while the compositions contain solid components, they also contain liquid materials. During wear of an absorbent article described herein, it is desirable to achieve a low viscosity for obtaining sufficient lubrication between the wearer's skin and the body contacting surface of the article to result in effective transfer of the lotion composition onto the wearer's skin. Viscosity at various shear stress can be measured using rheometers known in the art such as the Rheometer SR-2000 available from Rheometrics Incorporation.

The lotion compositions are typically applied to the topsheet of an absorbent article for delivery of the lotion composition onto an external or internal surface of the body. The lotion composition can be applied to other areas of the absorbent article wherein these areas include wings, side panels, the absorbent core, any secondary layer intermediate the core and topsheet, or any other region of the absorbent article.

Processes for assembling absorbent articles such as the disposable absorbent articles described herein include conventional techniques known in the art for constructing and configuring disposable absorbent articles. For example, the backsheet and/or the topsheet can be joined to the absorbent core or to each other by a uniform continuous layer of adhesive, a patterned layer of adhesive, or an array of separate lines, spirals, or spots of adhesive. Adhesives which have been found to be satisfactory are manufactured by H. B. Fuller Company of St. Paul, Minn. under the designation HL-1258 or H-2031.

### Test Methods

### Particle Size Distribution

The particle size and particle size distribution of the particulate material herein may me determined by any suitable method known in the art, but for the present invention a laser diffraction particle size analyzer (e.g., the Horiba LA-910) is used to perform the particle size measurement.

In order to determine the particle size of the particulate material prior to incorporation into the composition, the particles can be submitted to the method outlined below with the exception that the extraction is not required.

In order to determine the particle size of the particulate material in the composition prior to application thereof to the absorbent article, the composition can be submitted to the method outlined below.

To determine the particle size of the particulate material on the absorbent article, the particulate material needs to be removed from the absorbent article by any suitable extraction method, and then submitted to the method outlined below. For example, the topsheet or cuff will be removed from the article and then placed in a extraction liquid that does not dissolve particles, for example 70:30 ethanol:distilled water solution (some centrifuging or shaking may be needed to ensure the particles migrate and disperse into the solution. This may be repeated one or two times.

The resulting particles may then be isolated and submitted to the particle size measurements below.

### Laser Diffraction Particle Size Analyses

For this test, a Horiba LA-910 as is available from Horiba Europe GmbH of Sulzbach/Ts., Germany may be used according to the manufacturer's instructions, with the following set up conditions: Agitation speed: 2, Circulation speed: 1, Ultra Sonic speed: 1 min 0, Sampling times: 15. (The system should be confirmed for set-up and cleanliness by conducting a “Blank” particle size measurement of a known particulate material with known particle size distribution.)

A solution is chosen in which the articles do not dissolve but disperse easily, e.g. deionized water.

The sample to be tested is added dropwise using a disposable pipette, until the transmission (magenta bar) and backscatter (blue bar) indicator bars are in the green range of the display of the attached computer. This indicates that the particulate concentration in the sample chamber is high enough for a reliable measurement. Then, the software to conduct a particle size measurement is started. When the measurement is complete the display will show a report that includes a curve representing the particle size distribution and characteristics of the distribution (e.g., mean particle size).

The measurement is repeated to obtain two values and the average is herein reported as particle sizes and distributions.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

All documents cited in the Detailed Description of the Invention are, in relevant part, incorporated herein by reference; the citation of any document is not to be construed as an admission that it is prior art with respect to the present invention.

While particular embodiments of the present invention have been illustrated and described, it would be apparent to those skilled in the art that various other changes and modifications can be made without departing from the spirit and scope of the invention. It is therefore intended to cover in the appended claims all such changes and modifications that are within the scope of this invention.

What is claimed is:

1. An absorbent article, to be worn by a user, comprising a backsheet and a topsheet and/or leg cuffs and/or barrier cuffs, said topsheet, leg cuffs and/or barrier cuffs having a first surface, comprising a (partial) coating containing particulate material, obtained by applying a fluid or semi-fluid composition that comprises a fluid or semi-fluid carrier component and therein dispersed a particulate material.

2. An absorbent article as in claim 1 whereby said surface or surfaces is/are in use in contact with the skin of the user.

3. An absorbent article as in claim 1, whereby said particulate material is insoluble in water and insoluble in ethanol.

4. An absorbent article according to claim 1, whereby said particulate material prior to application to the topsheet or cuffs has a mean particle size from 20 to 1000 microns.

5. An absorbent article according to claim 1, whereby said composition is a skin care lotion composition that is semi-solid or solid at 20° C.

6. An absorbent article according to claim 5, whereby said lotion composition comprises 10% to 95% by weight of an emollient that has a plastic or fluid consistency at 20° C.

7. An absorbent article as in claim 6, whereby said lotion composition comprises 5% to about 90% by weight of an agent capable of immobilizing said emollient on said first surface, said immobilizing agent having a melting point of at least about 40° C.

8. An absorbent article as in claim 7, whereby said immobilizing agent is selected from the group consisting of polyhydroxy fatty acid esters, polyhydroxy fatty acid amides, C₁₄-C₂₂ fatty alcohols, C₁₂-C₂₂ fatty acids, C₁₂-C₂₂ fatty alcohol ethoxylates, and mixtures thereof.

9. An absorbent article according to claim 1, wherein said particulate material is present in said composition at a level of 0.1 to 10%.

10. An absorbent article according to claim 1, wherein said particulate material comprises particles of a, preferably single, organic material, preferably selected from the group consisting of polyolefin powders, polymethyl methacrylate microspheres, lactone cross polymer microspheres, nylon particles polymethylsilsesquioxane particles, cellulose particles.

11. An absorbent article according to claim 10, wherein said particulate material is selected from the group consisting of polyolefin powders, including polyethylene powder, polymethylsilsesquioxane particles and mixtures thereof.

12. An absorbent article according to claim 1, wherein said particulate material comprises particles of, preferably a single, inorganic material.

13. An absorbent article according to claim 1, whereby said coating composition comprises from 0.1 to about 50% of a hydrophilic surfactant, said hydrophilic surfactant having an HLB value of from about 4 to about 20.

14. An absorbent article according to claim 1, which is an infant or adult diaper, sanitary napkin or panty-liner, comprising at least said topsheet with said (partial) coating.

15. An absorbent article as in claim 14, whereby said topsheet comprises regions with said coating and regions without said coating.

16. Use in an absorbent article, for collection of feces and or menses and to be worn by a user, of a coating composition applied on a first surface of a topsheet, barrier cuff and/or leg cuff of said article, said first surface(s) being in contact with the skin of the user, said composition comprising a particulate material dispersed in a carrier component, to facilitate the removal of feces or menses from the skin of the user after use.

17. Use in an absorbent article, for collection of feces and or menses and to be worn by a user, of a coating composition applied on a first surface of a topsheet, barrier cuff and/or leg cuff of said article, said first surface(s) being in contact with the skin of the user, said composition comprising a particulate material dispersed in a carrier component, to reduce the deposition of feces or menses on the skin of the user.

18. Process for making an absorbent article of claim 1, comprising the steps of:
a) dispersing a particulate material in a fluid carrier component to obtain a fluid composition comprising particulate material;
b) applying this composition to the outer surface of a topsheet, barrier cuff and/or leg cuff or part thereof, form a coating or partial coating comprising particulate material.
